(19) United States
(12) Patent Application Publication
(10) Pub. No.: US 2023/0245376 A1
(43) Pub. Date: Aug. 3, 2023

(54) SYSTEM AND METHOD FOR FOUR-DIMENSIONAL ANGIOGRAPHY

(71) Applicant: Surgical Theater, Inc., Los Angeles, CA (US)

(72) Inventors: MORDECHAI AVISAR, HIGHLAND HEIGHTS, OH (US); Alon Yakob Geri, Orange Village, OH (US); DENNIS ROY, Irvine, CA (US); DAVID VIZMEG, PENINSULA, OH (US)

(73) Assignee: SURGICAL THEATER, INC., Los Angeles, CA (US)

(21) Appl. No.: 17/624,306

(22) PCT Filed: Jul. 14, 2021
(86) PCT No.: PCT/US2021/041567

§ 371 Date: Dec. 31, 2021

Related U.S. Application Data
(60) Provisional application No. 63/051,747, filed on Jul. 14, 2020.

(51) Int. Cl. G06T 15/08

(52) U.S. Cl. CPC G06T 15/08; G06T 2210/41

(57) ABSTRACT

A system and method for utilizing MD6DM models built from a SNAP case to generate an interactive four-dimensional (“4D”) angiogram, the 4th dimension including the dimension of time. By introducing such a 4th dimension to the angiogram, the example systems and methods enable visualization of a patient anatomy, such as blood flow over time, to be presented to a user in an interactive manner. This allows a user, such as a surgeon, to more effectively visualize and determine, for example, various anatomical features, such as the structure of an arteriovenous malformation (“AVM”).

### CROSS-REFERENCES TO RELATED APPLICATIONS

This application claims the benefit of U.S. Provisional Patent Application Ser. No. 63/051,747 filed on Jul. 14, 2020, incorporated herein by reference.

### BACKGROUND

Visualizing a blood vessel within a person's anatomy may aid in diagnosing and treating certain medical conditions, such as a stroke, an aneurysm, arteriovenous malformations, and blood clots, for example. A two-dimensional angiogram is a commonly used diagnostic imaging technique for enabling visualization of a blood vessel inside an anatomy for such diagnosis and treatment. A dye or a contrast agent is inserted into the blood vessels before a two-dimensional image, such as an X-ray or a CT scan, is taken of the anatomy. Because of the contrast agent, the blood vessel becomes visible in a scan or image.

A two-dimensional angiogram, however, may not always provide the necessary visualization of the blood vessels for effectively diagnosing and treating respective medical conditions. For example, certain portions of a blood vessel may not be visible from a given angle.

In one example, a surgery rehearsal and preparation tool previously described in U.S. Pat. No. 8,311,791 that has been developed to convert static medical images into dynamic and interactive multi-dimensional full spherical virtual reality, six (6) degrees of freedom models (“MD6DM”) and that can be used by physicians to simulate medical procedures in real time can also be used to create an angiogram.

The MD6DM provides a graphical simulation environment which enables the physician to experience, plan, perform, and navigate the intervention in full spherical virtual reality environment. In particular, the MD6DM gives the surgeon the capability to navigate using a unique multidimensional model, built from traditional 2 dimensional patient medical scans, that gives spherical virtual reality 6 degrees of freedom (i.e. linear; x, y, z, and angular, yaw, pitch, roll) in the entire volumetric spherical virtual reality model.

The MD6DM is built from the patient's own data set of medical images including CT, MRI, DTI etc. (a “SNAP case”), and is patient specific. A representative brain model, such as Atlas data, can be integrated to create a partially patient specific model if the surgeon so desires. The model gives a 360° spherical view from any point on the MD6DM. Using the MD6DM, the viewer is positioned virtually inside the anatomy and can look and observe both anatomical and pathological structures as if he were standing inside the patient's body. The viewer can look up, down, over the shoulders etc., and will see native structures in relation to each other, exactly as they are found in the patient. Spatial relationships between internal structures are preserved, and can be appreciated using the MD6DM.

The algorithm of the MD6DM takes the medical image information and builds it into a spherical model, a complete continuous real time model that can be viewed from any angle while “flying” inside the anatomical structure. In particular, after the CT, MM, etc. takes a real organism and deconstructs it into hundreds of thin slices built from thousands of points, the MD6DM reverts it to a 3D model by representing a 360° view of each of those points from both the inside and outside.

A 3D-360° view angiogram may be created using two-dimensional slices or images to provide a more complete visualization of an anatomy and the blood vessels. Nevertheless, the usefulness of the three-dimensional angiogram for diagnostic and treatment purposes may be limited in that visualization only represents a single snapshot in time of the blood vessels.

### SUMMARY

Provided are a plurality of example embodiments, including, but not limited to, a method for rendering an interactive model of a patient using a computer system, comprising the steps of:

the computer system receiving a first data set comprising representations of an anatomy of a patient at a first time period;

the computer system receiving a second data set comprising representations of the anatomy of the patient at a second time period different than the first time period, said second data set showing changes in the anatomy of the patient that occur over time;

the computer system generating a first virtual biological model of the anatomy of the patient using the first data set;

the computer system generating a second virtual biological model of the anatomy of the patient using the second data set, said second biological model showing the changes in the anatomy of the patient between the first time period and the second time period;

detecting a position and/or viewing direction of a user;

displaying the first virtual biological model to the user based on a perspective of a detected position and/or viewing direction of the user; and

subsequent to displaying the first virtual biological model to the user, displaying the second virtual biological model to the user based on the detected position and/or viewing direction of the user such that a transition of the display of the first virtual biological model to the second biological model is provided with a substantially seamless transition between models from the same perspective.

Also provided is a method for rendering an interactive model of a patient using a computer system, comprising the steps of:

the computer system receiving a plurality of data sets, each one of said data sets comprising representations of a 360° representation of the anatomy of a patient, each one of said data sets being taken at a different time period from all others of said data sets;

the computer system generating a plurality of biological models of the anatomy of the patient, each one of the plurality of biological models using a different one of the plurality of data sets, each one of said biological models showing the changes in the anatomy of the patient between different time periods of the plurality of data sets;

detecting a position and/or viewing direction of a user; and

displaying the plurality of virtual biological models to the user in sequence based on the detected position and/or viewing direction of the user such that a transition of the display between the different virtual biological models is provided in a substantially seamless manner between models from the same perspective in the chronological order of the time periods.

Further provided is a computer system for performing any of the above methods.

Also provided are additional example embodiments, some, but not all of which, are described hereinbelow in more detail.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates an example 4D angiogram system.

FIG. 2A illustrates an example output generated by the example 4D angiogram system of FIG. 1.

FIG. 2B illustrates an example output generated by the example 4D angiogram system of FIG. 1.

FIG. 3 illustrates an example 4D angiogram method.

FIG. 4 illustrates an example computer implementing the example 4D angiogram computer of FIG. 1.

### DETAILED DESCRIPTION

The following acronyms and definitions will aid in understanding the detailed description:

VR—Virtual Reality—A 3Dimensional computer generated environment which can be explored and interacted with by a person in varying degrees.

HMD—Head Mounted Display (FIG. 26), refers to a headset which can be used in AR or VR environments. It may be wired or wireless. It may also include one or more add-ons such as headphones, microphone, HD camera, infrared camera, hand trackers, positional trackers etc.

Controller—A device which includes buttons and a direction controller. It may be wired or wireless. Examples of this device are Xbox gamepad, PlayStation gamepad, Oculus touch, etc.

SNAP Case—A SNAP case refers to a 3D texture or 3D objects created using one or more scans of a patient (CT, MR, fMR, DTI, etc.) in DICOM file format. It also includes different presets of segmentation for filtering specific ranges and coloring others in the 3D texture. It may also include 3D objects placed in the scene including 3D shapes to mark specific points or anatomy of interest, 3D Labels, 3D Measurement markers, 3D Arrows for guidance, and 3D surgical tools. Surgical tools and devices have been modeled for education and patient specific rehearsal, particularly for appropriately sizing aneurysm clips.

Scene—Refers to the 3D virtual space, which includes the 3D texture and the 3D objects in it.

MD6DM—Multi Dimension full spherical virtual reality, 6 Degrees of Freedom Model. It provides a graphical simulation environment which enables the physician to experience, plan, perform, and navigate the intervention in full spherical virtual reality environment.

Described herein are example systems and methods that leverage the MD6DM model built from a SNAP case to generate an interactive angiogram which provides a view extending beyond a single snapshot in time of the blood vessels. In particular, the example systems and methods described herein leverage the MD6DM model built from a SNAP case to generate an interactive four-dimensional (“4D”) angiogram, the 4^{th} dimension including the dimension of time. By introducing such a 4^{th} dimension to the angiogram, the example systems and methods enable visualization of blood flow over time. This allows a user, such as a surgeon to more effectively visualize and determine, for example, the structure of an arteriovenous malformation (“AVM”). More specifically, it enables a physician to more easily and effectively differentiate between arteries and veins and how blood is flowing into and draining from the AVM. This enables the physician to more accurately and safely perform a surgical procedure, and in particular to determine how to remove the AVM. The 4D angiogram can be further used to engage and educate patients about their vascular system as blood flow may be difficult to demonstrate to a patient using known imaging tools. The 4D angiogram may also help a interventional radiologist better plan for various surgical procedures by enabling visualization of blood flow. It should be appreciated that aside from visualizing blood flow, the ability to more effectively distinguish arteries from veins using the 4D angiogram may also be beneficial for a variety of medical applications.

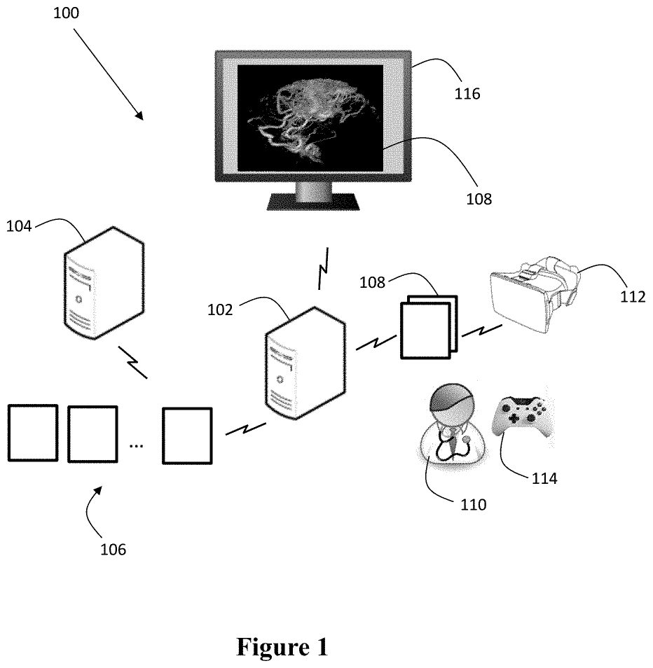

FIG. 1 illustrates an example 4D angiogram system 100. The 4D angiogram system 100 includes a 4D angiogram computer 102 that is configured to communicate with and receive as input MD6DM models 106 from a SNAP computer 104, such as a SNAP computer previously described in U.S. Pat. No. 8,311,791. It should be appreciated that although the 4D angiogram computer 102 is illustrated as being a standalone computer and separate from the SNAP computer 102, the functionality of the SNAP computer 102 and the 4D angiogram computer 102 may also be combined into a single computing system (not illustrated), in one example.

In order to facilitate generating a 4D angiogram, the 4D angiogram computer 102 receives from the SNAP computer 102 multiple data sets or MD6DM models 106 of a patient's anatomy at different times stamps. The 4D angiogram computer 102 is then configured to seamlessly integrate the multiple data sets 106 into a single 4D model 108 that a physician 110 may interact with in order to, for example, experience and visualize blood flow over time through an anatomy. It should be appreciated that although specific reference is made herein with respect to using the 4D model 108 as a 4D angiogram for visualizing blood flow over time, the 4D model can be used for other suitable purposes and for visualizing other changes in the anatomy over time that can be identified in the data sets 106.

In one example, the 4D angiogram computer 102 is configured to communicate the 4D angiogram 108 to a head mounted display (“HMD”) 112, thereby enabling the physician 110 to interact with the 4D angiogram 108 using an associated controller 114 or other similar input device. In particular, the physician 110 is able to freely fly through or explore the anatomy represented by the data sets 106 virtually and visualize how the model is changing over time as the 4D angiogram computer 102 renders changes to the 4D angiogram 108 over time. In another example, the 4D angiogram computer 102 may communicate the 4D angiogram 108 to any suitable display 116.

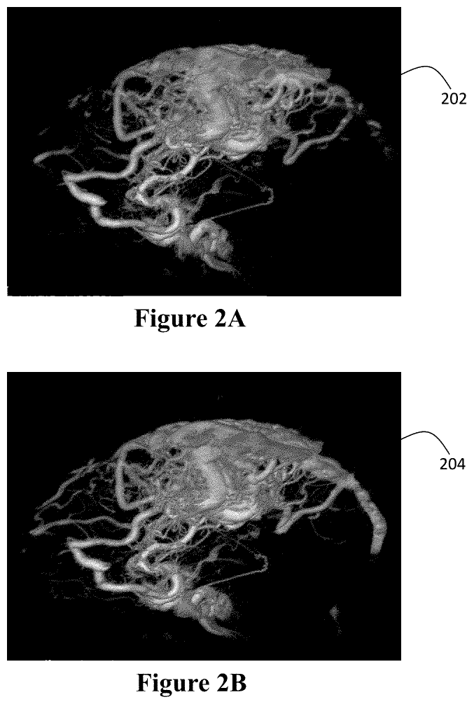

FIG. 2A illustrates a first closeup view 202 of the 4D angiogram 108 rendered at a first point in time or time stamp while 2B illustrates a second closeup view 204 of the 4D angiogram 108 rendered at a second point in time or time stamp. By “flipping” from a first time stamp to a second time stamp, a user or physician is able to see the changes in the blood vessels, thereby experiencing motion of blood over time.

Referring back to FIG. 1, it should be appreciated that the 4D angiogram computer 102 is able to integrate or fuse together any suitable number of data sets 106 or time stamps. For example, in a most simple form, the 4D angiogram computer 102 fuses together data sets 106 including 2 samples or time stamps in order to provide a view of the change in blood vessels between a first time and a second time. However, as can be appreciated, fusing more time stamps or samples of data creates a more realistic view of the flow of blood over time. Thus, in one example, the 4D angiogram computer 102 is configured to fuse together ten time stamps. In another example, the 4D angiogram computer 102 is configured to fuse together one hundred time stamps.

In order to seamlessly fuse together multiple time stamps or MD6DM models/data sets 106 and allow a user to freely interact with the resulting 4D angiogram 108, the 4D angiogram computer 102 tracks the physician's 110 virtual position as the physician 110 freely navigates within a single MD6DM model of the data set 106. Prior to flipping or switching views to a new time stamp or MD6DM model, the 4D angiogram computer 102 records the physician's virtual location within the model. After the flip, the 4D angiogram computer 102 seamlessly positions the physician 110 virtually within the same location inside the model as previously left off. Thus, from the physician's 110 perspective, the view, including the location, direction, and angle of view, does not change in that instant, but rather the only thing that changes is the time during which the physician is observing the anatomy or blood vessels. Thus, the physician 110 experiences a simulated change in view of a blood vessels over time from the same location.

It should be appreciated that such a seamless flip or transition point between two different MD6DM models at different times stamps will occur one time if the data set 106 includes two time stamps or samples. However, additional flips or transitions will occur for each additional time stamp or sample of data included in the data set 106. For example, a data set with 100 time stamps will require the 4D angiogram computer 102 to execute 99 transitions while rendering the resulting 4D angiogram 108.

The 4D angiogram computer 102 is configured to wait a specified amount of time or a delay in between flipping between a time stamp. For example, for a data set 106 having a small sample size, the 4D angiogram computer 102 may use a longer delay for rendering the 4D angiogram while the 4D angiogram computer 102 may use a shorter delay for rendering the 4D angiogram using a data set 106 with a larger sample size. In one example, the delay may be configurable by the physician 110 or other user. Furthermore, the flipping operation may be provided in a real-time sequence (but not live and not necessarily in a smooth, continuous motion if the delay between data sets is larger), or sped up, or slowed down, as desired. The sequence can also be reversed in time if desired.

In one example, the 4D angiogram computer 102 is configured to execute the flips between time stamps according to a delay that tracks the actual delay between when the samples of the data set 106 were captured (i.e., shows the sequences in real-time). For example, a data set 106 may include a sample size of 100 MD6DM models that were constructed from data or images captured once every 5 seconds. Accordingly, the 4D angiogram computer 102 may be configured to render the resulting 4D angiogram based on that data set 106 using a 5 second delay. Alternatively, delays can run from fractions of a second (such as to provide real-time continuous imaging in some cases such as where delays are 1/30 of a second, for example), to one second, two seconds, or any other desired value.

In one example, the 4D angiogram computer 102 may be configured to loop or repeat the time stamps continuously while rendering the 4D angiogram 108. For example, after the 4D angiogram computer 102 has rendered the final MD6DM model of the data set 106 and the final delay has expired, the 4D angiogram computer 102 may again render the first MD6DM model in the data set 106 and repeat the cycle. This may be desirable, for example, when the data set 106 has a limited number of time stamps and the physician 110 still wants to experience continuous movement of the anatomy or blood vessels.

In one example, the 4D angiogram computer 102 may be configured to aid in differentiating blood vessels by assigning different colors to veins and arteries. Thus, the color coding of blood vessels in combination visualizing the flow of blood within the vessels over time may further aid a physician in performing various medical procedures. In one example, the 4D angiogram computer 102 is configured to differentiate between blood vessels by comparing the blood vessels over time. In particular, by observing and comparing the changes in intensity in the blood vessels over time as the 4D angiogram computer 102 fuses together the different data sets 106, the 4D angiogram computer 102 is able to label a blood vessel as an artery or a vein and to assign an appropriate color accordingly.

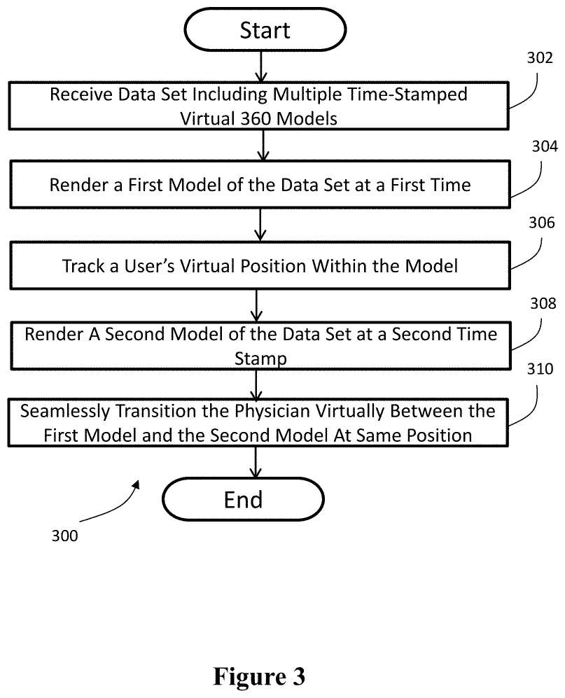

FIG. 3 illustrates an example method 300 for rendering an interactive 4D angiogram. At 302, the 4D angiogram computer 102 receives a data set including multiple time stamped virtual 360 representations of a patient's anatomy. At 304, the angiogram computer 102 renders a first time stamped virtual 360 representation of the patient's anatomy for a physician to view and freely interact with at a first time. At step 306, the 4D angiogram computer 102 tracks and records the physician's position within the first rendered virtual 360 representation. At 308, the angiogram computer 102 renders a second time stamped virtual 360 representation of the patient's anatomy for the physician to view and freely interact with at a second time. At step 310, the angiogram computer 102 seamlessly transitions the physician virtually between the first time stamped virtual 360 representation and the second time stamped virtual 360 representation at the same virtual location.

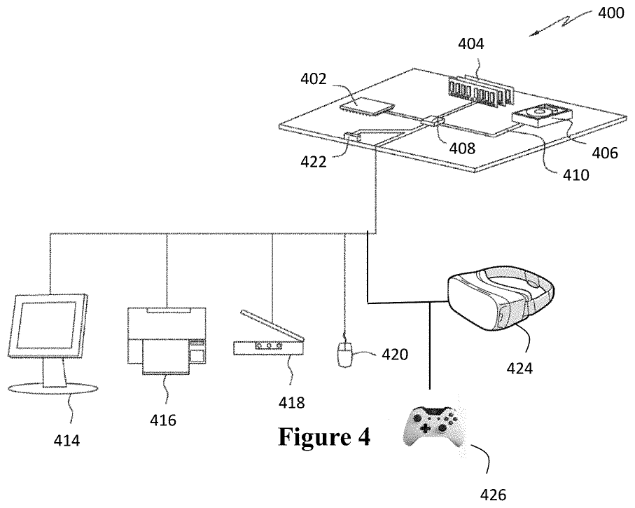

FIG. 4 is a schematic diagram of an example computer for implementing the 4D angiogram computer 102 of FIG. 1. The example computer 400 is intended to represent various forms of digital computers, including laptops, desktops, handheld computers, tablet computers, smartphones, servers, AR glasses, and other similar types of computing devices. Computer 400 includes a processor 402, memory 404, a storage device 406, and a communication port 408, operably connected by an interface 410 via a bus 412.

Processor 402 processes instructions, via memory 404, for execution within computer 800. In an example embodiment, multiple processors along with multiple memories may be used.

Memory 404 may be volatile memory or non-volatile memory. Memory 404 may be a computer-readable medium, such as a magnetic disk or optical disk. Storage device 406 may be a computer-readable medium, such as floppy disk devices, a hard disk device, optical disk device, a tape device, a flash memory, phase change memory, or other similar solid state memory device, or an array of devices, including devices in a storage area network of other configurations. A computer program product can be tangibly embodied in a computer readable medium such as memory 404 or storage device 406.

Computer 400 can be coupled to one or more input and output devices such as a display 414, a printer 416, a scanner 418, a mouse 420, a, HMD 424, and a controller 426.

As will be appreciated by one of skill in the art, the example embodiments may be actualized as, or may generally utilize, a method, system, computer program product, or a combination of the foregoing. Accordingly, any of the embodiments may take the form of specialized software comprising executable instructions stored in a storage device for execution on computer hardware, where the software can be stored on a computer-usable storage medium having computer-usable program code embodied in the medium.

Databases may be implemented using commercially available computer applications, such as open source solutions such as MySQL, or closed solutions like Microsoft SQL that may operate on the disclosed servers or on additional computer servers. Databases may utilize relational or object oriented paradigms for storing data, models, and model parameters that are used for the example embodiments disclosed above. Such databases may be customized using known database programming techniques for specialized applicability as disclosed herein.

Any suitable computer usable (computer readable) medium may be utilized for storing the software comprising the executable instructions. The computer usable or computer readable medium may be, for example but not limited to, an electronic, magnetic, optical, electromagnetic, infrared, or semiconductor system, apparatus, device, or propagation medium. More specific examples (a non-exhaustive list) of the computer readable medium would include the following: an electrical connection having one or more wires; a tangible medium such as a portable computer diskette, a hard disk, a random access memory (RAM), a read-only memory (ROM), an erasable programmable read-only memory (EPROM or Flash memory), a compact disc read-only memory (CDROM), or other tangible optical or magnetic storage device; or transmission media such as those supporting the Internet or an intranet.

In the context of this document, a computer usable or computer readable medium may be any medium that can contain, store, communicate, propagate, or transport the program instructions for use by, or in connection with, the instruction execution system, platform, apparatus, or device, which can include any suitable computer (or computer system) including one or more programmable or dedicated processor/controller(s). The computer usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, local communication busses, radio frequency (RF) or other means.

Computer program code having executable instructions for carrying out operations of the example embodiments may be written by conventional means using any computer language, including but not limited to, an interpreted or event driven language such as BASIC, Lisp, VBA, or VBScript, or a GUI embodiment such as visual basic, a compiled programming language such as FORTRAN, COBOL, or Pascal, an object oriented, scripted or unscripted programming language such as Java, JavaScript, Perl, Smalltalk, C++, C#, Object Pascal, or the like, artificial intelligence languages such as Prolog, a real-time embedded language such as Ada, or even more direct or simplified programming using ladder logic, an Assembler language, or directly programming using an appropriate machine language.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

What is claimed is:

1. A method for rendering an interactive model of a patient using a computer system, comprising the steps of:
the computer system receiving a first data set comprising representations of an anatomy of a patient at a first time period;
the computer system receiving a second data set comprising representations of the anatomy of the patient at a second time period different than the first time period, said second data set showing changes in the anatomy of the patient that occur over time;
the computer system generating a first virtual biological model of the anatomy of the patient using the first data set;
the computer system generating a second virtual biological model of the anatomy of the patient using the second data set, said second biological model showing the changes in the anatomy of the patient between the first time period and the second time period;
detecting a position and/or viewing direction of a user;
displaying the first virtual biological model to the user based on a perspective of a detected position and/or viewing direction of the user; and
subsequent to displaying the first virtual biological model to the user, displaying the second virtual biological model to the user based on the detected position and/or viewing direction of the user such that a transition of the display of the first virtual biological model to the second biological model is provided with a substantially seamless transition between models from the same perspective.

2. The method of claim 1, wherein said virtual models are interactive 4D MD6DM models of the patient supporting a 360° representation of the anatomy of the patient.

3. The method of claim 2, wherein said first and second data sets are of 3D angiograms.

4. The method of claim 1, wherein said first and second data sets are of 3D angiograms.

5. The method of claim 1, wherein said second time period is one or more seconds after said first time period.

6. The method of claim 1, wherein said second time period is about five seconds after said first time period.

7. The method of claim 1, wherein said second time period is a fraction of a second after said first time period.

8. The method of claim 1, further comprising the steps of:
the computer system receiving a plurality of additional data sets, each one of said additional data sets comprising representations of the anatomy of a patient and each one of said additional data sets being taken at a different time period from all others of said data sets;
the computer system generating additional biological models of the anatomy of the patient, each one of the additional biological models using a different one of the additional data sets, said additional biological models showing the changes in the anatomy of the patient between the different time periods of the additional data sets; and
displaying the additional virtual biological models to the user in a time sequence, said displaying based on the detected position and/or viewing direction of the user such that a transition of the display between the different virtual biological models is also provided in a substantially seamless manner between models from the same perspective.

9. The method of claim 8, further comprising the step of displaying the virtual biological models in reverse order.

10. The method of claim 8, further comprising the step of displaying the virtual biological models in a repeating loop.

11. The method of claim 8, wherein said virtual models are interactive 4D MD6DM models of the patient supporting a 360° representation of the anatomy of the patient.

12. The method of claim 1, wherein blood vessels of the anatomy of the patient in said virtual models are displayed different colors assigned to veins as opposed to arteries.

13. The method of claim 1, wherein said the changes in the anatomy of the patient include changes in the blood vessels of the patient.

14. A method for rendering an interactive model of a patient using a computer system, comprising the steps of:
the computer system receiving a plurality of data sets, each one of said data sets comprising representations of a 360° representation of the anatomy of a patient, each one of said data sets being taken at a different time period from all others of said data sets;
the computer system generating a plurality of biological models of the anatomy of the patient, each one of the plurality of biological models using a different one of the plurality of data sets, each one of said biological models showing the changes in the anatomy of the patient between different time periods of the plurality of data sets;
detecting a position and/or viewing direction of a user; and
displaying the plurality of virtual biological models to the user in sequence based on the detected position and/or viewing direction of the user such that a transition of the display between the different virtual biological models is provided in a substantially seamless manner between models from the same perspective in the chronological order of the time periods.

15. The method of claim 14, wherein said interactive model is an interactive 4D MD6DM model of the patient.

16. The method of claim 14, wherein said first and second data sets are of 3D angiograms.

17. The method of claim 14, wherein said time periods are one or more seconds apart from each other.

18. The method of claim 14, wherein said time periods are about five seconds apart from each other.

19. The method of claim 14, wherein said time periods are a fraction of a second apart from each other.

20. The method of claim 14, further comprising the step of displaying the virtual biological models in reverse order.

21. The method of claim 14, further comprising the step of displaying the virtual biological models in a repeating loop.

22. The method of claim 14, wherein blood vessels of the anatomy of the patient in said virtual models are displayed different colors assigned to veins as opposed to arteries.

23. The method of claim 14, wherein said the changes in the anatomy of the patient include changes in the blood vessels of the patient.

24. A system for rendering an interactive model of a patient using a computer system, comprising a computer system including software configured to perform the steps of:
the computer system receiving a plurality of data sets, each one of said data sets comprising representations of a 360° representation of the anatomy of a patient, each one of said data sets being taken at a different time period from all others of said data sets;
the computer system generating a plurality of biological models of the anatomy of the patient, each one of the plurality of biological models using a different one of the plurality of data sets, each one of said biological models showing the changes in the anatomy of the patient between different time periods of the plurality of data sets;
detecting, using a sensor, a position and/or viewing direction of a user; and
displaying, using a display, the plurality of virtual biological models to the user in sequence based on the detected position and/or viewing direction of the user such that a transition of the display between the different virtual biological models is provided in a substantially seamless manner between models from the same perspective in the chronological order of the time periods.

25. The system of claim 24, wherein said interactive model an interactive 4D MD6DM model of the patient.

26. The system of claim 24, wherein said first and second data sets are of 3D angiograms.

27. The system of claim 24, further comprising the step of displaying the virtual biological models in reverse order.

28. The system of claim 24, further comprising the step of displaying the virtual biological models in a repeating loop.

29. The system of claim 24, wherein blood vessels of the anatomy of the patient in said virtual models are displayed different colors assigned to veins as opposed to arteries.

30. The system of claim 24, wherein said the changes in the anatomy of the patient include changes in the blood vessels of the patient.
